(19) United States
(12) Patent Application Publication
(10) Pub. No.: US 2016/0083522 A1
(43) Pub. Date: Mar. 24, 2016

(54) Arginine-Grafted Bioreducible Polymer Systems and Use in Treatment of Cardiac Conditions

(71) Applicant: UNIVERSITY OF UTAH RESEARCH FOUNDATION, Salt Lake City, UT (US)

(72) Inventors: Sung Wan Kim, Salt Lake City, UT (US); Young Sook Lee, Salt Lake City, UT (US); Minhyung Lee, Gwacheon-city (KR)

(73) Assignee: UNIVERSITY OF UTAH RESEARCH FOUNDATION, Salt Lake City, UT (US)

(21) Appl. No.: 14/891,289

(22) PCT Filed: May 14, 2014
(86) PCT No.: PCT/US2014/000112

§ 371 Date: Nov. 13, 2015

Related U.S. Application Data
(60) Provisional application No. 61/855,374, filed on May 14, 2013.

(51) Int. Cl. C08G 75/14; A61K 31/795

(52) U.S. Cl. CPC C08G 75/14; A61K 31/795

(57) ABSTRACT

phEPO/ABP polyplexes and methods for the use thereof are disclosed and described. In one embodiment, a phEPO/ABP polyplex may be administered to a subject in a therapeutically effective amount to treat or prevent a cardiac condition. Administration may 5 be made, in some aspects, by intramyocardial injection of a composition or solution containing the phEPO/ABP polyplex.

### PRIORITY DATA

This application claims the benefit of U.S. Provisional Patent Application No. 61/855,734, filed May 14, 2013 which is incorporated herein by reference.

### STATEMENT REGARDING FEDERAL SUPPORT

This invention was made with government support under grant no. HL065477 from the National Institute of Health. The United States government has certain rights in the invention.

### BACKGROUND

Despite remarkable advances in guideline-based pharmacologic and interventional treatment over the last two decades, myocardial infarction (MI) is the leading cause of morbidity, and mortality worldwide. The post-infarcted heart undergoes a series of structural changes, termed left ventricular (LV) remodeling, at the organ, cellular, and molecular levels, with three overlapping phases: the inflammatory phase, the proliferative phase, and the healing phase. Although cardiac remodeling is initially an adaptive response to maintain normal cardiac function, it gradually becomes maladaptive and can lead to adverse clinical outcomes, including heart failure (HF), arrhythmia, and mortality. Diverse efforts in experimental and clinical trials have been made to investigate cardioprotective strategies aimed at attenuating reperfusion injury, reversing adverse myocardial remodeling, and ultimately improving cardiac systolic function and clinical outcomes.

During the last two decades, the clinical indications of recombinant human erythropoietin (rHuEPO) have been expanded to anemia in diverse clinical categories, including anemic patients with chronic kidney disease. Beyond the conventional effect of secreted erythropoietin from the kidney in response to hypoxic stimuli, erythropoietin (EPO) has been identified as a pleiotropic and organ-protective cytokine. It also is thought to mediate repair and regeneration via anti-apoptosis, act as an anti-inflammatory, and act as an anti-oxidant. It further is implicated in pro-angiogenesis and re-endothelialization, as a vascular-protectant, in mobilization of endothelial progenitor cells, and in recruitment of stem cells into the zone of damage.

The development of drug delivery systems (DDS) has provided new perspectives for the modification of pharmacokinetics and biodistribution of associated genes and proteins by controlling the release rates of therapeutics. One of the requirements for successful gene therapy is the development of non-toxic and efficient carriers for gene delivery. Compared to viral vectors, non-viral gene carriers such as lipids, synthetic polymers and/or peptides offer a number of advantages including easy and large-scale production, non-immunogenicity, flexible DNA and RNA loading capacity and stability among others. Despite these advantages, however, the widespread adoption of non-viral gene vectors has been limited by concerns related to cytotoxicity and decreased transfection efficiency. However, since the accumulation of non-degraded polymers inside cells is often the cause of cytotoxicity, the biodegradation of polymers after efficient transfection of DNA can reduce or eliminate this problem. Biodegradable polymers typically contain ester or disulfide-bonds. Ester bonds, however, are easily hydrolyzed in the extracellular environment; disulfide bonds are typically more stable, as they are not reduced until they are exposed to glutathione (GSH) in the intracellular cytoplasm.

### BRIEF DESCRIPTION OF THE DRAWINGS

Additional features and advantages of the disclosed embodiments will be apparent from the detailed description which follows, taken in conjunction with the accompanying drawings, which together illustrate, by way of example, features of the invention.

FIG. 1a shows a graphical representation of the characterization of phEPO/ABP polyplexes showing average particle size and zeta potential of 50 μg phEPO/ABP polyplex in a 1:5 w/w ratio according to an invention embodiment.

FIG. 1b shows a graphical representation of the characterization of phEPO/PEI polyplexes showing average particle size and zeta potential of 50 μg phEPO/PEI polyplex in a 1:1 w/w ratio according to an invention embodiment.

FIG. 1c shows an example of an experimental time protocol in accordance with an invention embodiment.

FIG. 2a shows a graphical representation of echocardiography measured parameters of cardiac geometry and function during post-infarct cardiac remodeling at 5 days after MI.

FIG. 2b shows a graphical representation of echocardiography measured parameters of cardiac geometry and function during post-infarct cardiac remodeling at 10 days after MI.

FIG. 3a shows representative Masson's trichrome staining images of tissue in the mid-ventricle of hearts from various groups tested in accordance with an invention embodiment.

FIG. 3b shows a graphical representation of the quantification of percent fibrosis area in left ventrical (LV) in test subjects tested in accordance with an invention embodiment.

FIG. 4a shows representative IHC staining images of tested tissue for cTnT in the mid-ventricle of hearts from tested groups tested in accordance with an invention embodiment.

FIG. 4b shows graphical quantification of percent cardiomyocytes loss in LV adjusted by the level of thoracotomy group tested in accordance with an invention embodiment.

FIG. 4c shows representative TUNEL staining images in the LVfb from each group tested in accordance with an invention embodiment.

FIG. 4d shows quantification of corrected TUNEL positive cells (mm2) corrected by the level of thoracotomy group tested in accordance with an invention embodiment.

FIG. 5a shows IHC staining images of tissue tested for α-SMA as a result of cardiac remodeling occurring in accordance with an invention embodiment.

FIG. 5b shows a graphical representation of a quantification of pro-angiocenic activity by the α-SMA-positive arterioles adjusted by the level of thoracotomy group tested in accordance with an invention embodiment.

FIG. 5c shows IHC staining images of tissue tested for distribution and density of myoFbs as a result of cardiac remodeling occurring in accordance with an invention embodiment.

FIG. 5d shows a graphical representation of a quantification of α-SMA-positive myoFb differentiation adjusted by the level of thoracotomy group tested in accordance with an invention embodiment.

FIG. 6a shows Fibrogenic Ang II expression in cardiac tissues between different treatment groups by Western blot analysis, according to the subdivision of cardic tissues—LVf, LVfb, RV, atria, and IVS, tested in accordance with an invention embodiment.

FIG. 6b shows TGF-β (B) expression in cardiac tissues between different treatment groups by Western blot analysis, according to the subdivision of cardic tissues—LVf, LVfb, RV, atria, and IVS, tested in accordance with an invention embodiment.

Reference will now be made to the exemplary embodiments illustrated, and specific language will be used herein to describe the same. It will nevertheless be understood that no limitation of the scope of the invention is thereby intended.

### DETAILED DESCRIPTION OF EMBODIMENTS

It is to be understood that this invention is not limited to the particular process steps and materials disclosed herein, but is extended to equivalents thereof, as would be recognized by those ordinarily skilled in the relevant arts. It should also be understood that terminology employed herein is used for the purpose of describing particular embodiments only and is not intended to be limiting.

It should be noted that, the singular forms “a,” “an,” and, “the” include plural referents unless the context clearly dictates otherwise. Thus, for example, reference to “a polymer” includes reference to one or more of such polymers, and reference to “the nucleic acid” includes reference to one or more of such nucleic acids.

### DEFINITIONS

Unless defined otherwise, all technical and scientific terms used herein have the same meanings as commonly understood by one of ordinary skill in the art to which this invention belongs.

In describing and claiming the present invention, the following terminology will be used in accordance with the definitions set out below.

As used herein, the term “about” is used when used in the context of a numerical range provides flexibility to the numerical range endpoint(s) by providing that a given value may be “a little above” or “a little below” the endpoint(s).

As used herein, “comprising,” “including,” “containing,” “characterized by,” and grammatical equivalents thereof are inclusive or open-ended terms that do not exclude additional, un-recited elements or method steps. As used in this specification, “comprising” is to be interpreted as including support for the more restrictive terms “consisting of” and “consisting essentially of,” and vice versa. As used herein, “consisting of” and grammatical equivalents thereof exclude any element, step, or ingredient not specified in the claim. As used herein, “consisting essentially of” and grammatical equivalents thereof limit the scope of a claim to the specified materials or steps and those that do not materially affect the basic and novel characteristic or characteristics of the claimed invention.

As used herein, a “complex” refers to a molecular entity formed by an association between at least two chemical components that is formed by a force other than covalent bonds and a “polyplex” refers to a complex of a polymer and DNA. These terms are well known in the relevant biological and chemical arts.

As used herein, the acronym “ABP” refers to arginine-conjugated bioreducible poly(disulfide amine) polymers which can be used, bonded to, or otherwise complexed or polyplexed with, nucleic acid or other biologic materials. One embodiment of an ABP may include the following general structure:

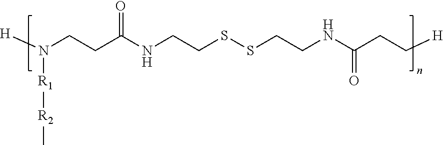

wherein n is about 1 to 1000 and wherein R₁ is (CH₂)ₘNH, wherein m is 1 to 18; and R₂ is an arginine residue. Illustratively, R₂ can comprise (CH₂) 6NH, (CH₂) 4NH, or (CH₂) 2NH, and a selected nucleic acid can comprise a plasmid (i.e. pDNA), siRNA, or an oligonucleotide. Examples of various ABP's and the preparation thereof are shown in U.S. patent application Ser. Nos. 12/267,015; 12/370,515; and 12/496,568; and in PCT Application Serial No. PCT/US13/22294, each of which are incorporated herein by reference.

As used herein, “phEPO” refers to a plasmid form of human erythropoietin gene. In one aspect the phEPO is a pCMV-hEPO cDNA molecule having 4,578 bp. An exemplary method for production of phEPO is described herein.

As used herein, “phEPO/ABP” refers to a complex or polyplex of phEPO with ABP, for example a cationic polyplex. An exemplary method for production of a phEPO/ABP is described herein.

As used herein, “nucleic acid,” “nucleic acid materials,” “nucleotides,” and the like may be used interchangeably and can refer to any type or form of nucleic acid material, including without limitation siRNA, plasmids (i.e. pDNA), complimentary DNA (i.e. cDNA), or oligonucleotides.

As used herein, “poly(CBA-DAH)” refers to polymers formed between cystaminebisacrylamide (“CBA”) and 1,6-diaminohexane (“DAH”). Similarly, “poly(CBA-DAB)” refers to polymers formed between CBA and 1,4-diaminobutane (“DAB”), and “poly(CBA-DAE)” refers to polymers formed between CBA and 1,2-diaminoethane (“DAE”).

As used herein, “PH” means polyethylenimine, “PEI25k” means polyethylenimine having a nominal molecular weight of about 25,000, and “bPEI” means branched polyethylenimine.

As used herein, “administering” and similar terms mean delivering a compound, complex, or polyplex to an individual being treated for a cardiac condition such that the compound, complex, or polyplex can contact and be internalized in cells, such as cardiac cells. Thus, in one embodiment the compound, complex, or polyplex can be administered to the individual by systemic administration, such as by subcutaneous, intramuscular, or intravenous administration, or intraperitoneal administration. In another aspect, the administration may be local, for example specifically and primarily to cardiac cells or tissue. Injectables for such use can be prepared in conventional forms, either as a liquid solution or suspension or in a solid form suitable for preparation as a solution or suspension in a liquid prior to injection, or as an emulsion. Suitable excipients/carriers include, for example, water, saline, dextrose, glycerol, ethanol, and the like; and if desired, minor amounts of auxiliary substances such as wetting or emulsifying agents, buffers, and the like can be added. Other known modes of administration can also be used including, but not limited to oral administration and transdermal administration for either local or systemic delivery.

As used herein, the term “treatment,” “treating,” and the like, when used in conjunction with the administration of phEPO/ABP, including in compositions and specific dosage forms, refers to the administration to subjects who are either asymptomatic or symptomatic. In other words, “treatment” and “treating” can be to reduce or eliminate symptoms associated with a condition present in a subject, or it can be prophylactic treatment, i.e. to prevent the occurrence of the symptoms in a subject. Such prophylactic treatment can also be referred to as prevention of the condition.

As used herein, the terms “formulation” and “composition” are used interchangeably and refer to a mixture of two or more compounds, elements, molecules, complexes, or polyplexes. In some aspects the terms “formulation” and “composition” may be used to refer to a mixture of phEPO/ABP with a carrier or other excipients. Furthermore, the term “dosage form” can include one or more formulation(s) or composition(s) provided in a format for administration to a subject.

As used herein, “subject” refers to a mammal that may benefit from the administration of a phEPO/ABP including composition containing such, or method of this invention. Examples of subjects include humans.

As used herein, an “effective amount” or a “therapeutically effective amount” of a phEPO/ABP refers to a non-toxic, but sufficient amount to achieve therapeutic results in treating a condition for which it is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “condition which is responsive to erythropoietin therapy,” or “condition in a subject which is responsive to erythropoietin therapy,” refers to any disease, state, condition, or ailment which benefits, improves, or is ameliorated by an increase in erythropoietin presence, levels, or concentration. Examples of such include without limitation, hypoxia (including any underlying cause), cardiac conditions, inflammatory bowel disease (Crohn's disease and ulcer colitis), anemia (including any underlying causality), kidney diseases or conditions (including chronic renal failure), physiologic conditions which negatively impact or reduce kidney performance, such as diabetes, or any other disease, state, condition, or ailment, which causes or contributes to a reduction in volume, output, or performance of red blood cells, including anemia associated cancers, or treatments for diseases, such as chemotherapy or radiation therapy.

As used herein, “cardiac condition(s)” refers to any condition affecting the heart or related tissue including vasculature within the heart. Such conditions may be acute or chronic and may be caused by a trauma, injury, or disease of cardiac tissue, such as myocardial infarction (MI), blunt force trauma, infection, inflammation, incision, or any other condition or event that diminishes, destroys, or adversely impacts cardiac function. Further, such conditions may be the result of the body's response to such trauma, injury, or disease, such as remodeling of cardiac tissue. In some embodiments, cardiac conditions may include one or more specific activities such as fibrosis, cardiomyocyte loss, as well as apoptotic activity.

As used herein, “cardiac cells,” “cardiac tissue,” and the like, refers to cells found in the heart organ. Generally, the heart includes several types of tissue, namely, endocardium myocardium, epicardium, and pericardium. A number of different cell types make up such tissues, such as for example, myocytes (i.e. cardiomyocytes), endothelial cells, and epithelial cells.

As used herein, “cardiac remodeling” refers to changes in cardiac tissue, structure, function, or other properties as a result of the process of healing or recovering from a cardiac condition. Cardiac remodeling can be adverse or negative (i.e. deleterious changes) or it can be beneficial or positive (i.e. helpful changes).

As used herein, “erythropoietic effect” refers to a positive or beneficial effect on a subject obtained by administering phEPO/ABP to the subject. In one aspect, the erythropoietic effect may be from erythropoietin expression induced by phEPO/ABP administration and resulting in an elevated level of erythropoiesis. Examples of positive or beneficial effects include without limitation, increased erythropoiesis (red blood cell production), stimulation of angiogenesis, neuroprotection, and proliferation of smooth muscle fibers.

As used herein, a “carrier” or a “pharmaceutically acceptable carrier” refers to an agent with which a phEPO/ABP polyplex as recited herein may be combined in order to form a composition or specific dosage form. Generally, such carriers are safe for administration to a subject without toxicity or other potential adverse effect when administered in an amount sufficient to perform as a carrier for the polyplex. A number of safe and effective carriers are known for various dosage forms. One example of such a carrier for parenteral administration is water, particularly deionized or filtered water. Other examples of ingredients that may be part of a carrier or may otherwise qualify as an excipient include buffers, tonicity agents, salts, sugars, such as glucose, and the like. In some aspects, carriers or excipients may improve the stability of a phEPO/ABP polyplex, or provide other administration benefits.

As used herein, “free of” or “substantially free of” of a particular compound or compositions refers to the absence of any separately added portion of the referenced compound or composition. Free of or substantially free of can include the presence of 1 wt % or less (based on total composition weight) of the referenced compound which may be present as a component or impurity of one or more of the ingredients.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Invention

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, variants, modifications, and equivalents as may be included within the spirit and scope of the invention as defined by the appended claims.

Nonviral gene therapy, especially using cationic polymers, provides great potential for human gene therapy due to capacity to carry large nucleic acid loads, biosafety with low immunogenicity, and easy modification. One embodiment the present invention provides ABP polymers and systems that retain the unique properties of reductive disulfide linkers coupled with the advantage of arginine residues to enhance cell penetration for use in treatment of cardiac conditions. It has been discovered that ABP-erythropoietin complexes/polyplexes have surprising positive effects on post MI cardiac remodeling and other cardiac conditions. For example, the plasmid human erythropoietin gene (phEPO) can be delivered by an ABP in order to produce cardioprotective effects during post-infarct cardiac remodeling. In some aspects, greatly enhanced in vitro transfection efficiency and very low cytotoxicity, as well as increased in vivo erythropoietic effects over a 60-day period after a single systemic injection of plasmid human erythropoietin phEPO/ABP polyplexes can be achieved. In one aspect, the injection may be made directly to cardiac tissue. In some embodiments, polymer-mediated phEPO therapy, when compared with naked phEPO gene or rHuEPO protein-alone, distinctly alters cardiac remodeling. EPO gene therapy delivered by ABP polymer augments the action of EPO compared to rHuEPO or phEPO alone. One possible mechanism for this is its prolonged stability in serum. This long-term erythropoietin expression is beneficial for treatment of both acute and long-term progressive conditions, for example, acute MI.

It is thought by the present inventors that the sustained release of intramyocardial phEPO gene therapy delivered by ABP polymer can restore heart function and limit pathological cardiac remodeling after MI. Cardiac geometry and systolic function can be preserved. Reduced infarct size of phEPO/ABP delivery is followed by decrease in fibrosis, protection from cardiomyocyte loss, and down-regulation of apoptotic activity. In addition, increased angiogenesis and decreased myofibroblast density in the border zone of the infarct support the beneficial effects of phEPO/ABP administration. One measure of phEPO/ABP gene therapy effect on cardiac remodeling may be made by evaluating the pro-fibrotic angiotensin II (Ang II) and TGF-β expression heart tissue. As elaborated further herein, administration of the present phEPO/ABP polyplexes induces prominent suppression on Ang II and TGF-β activity in all subdivisions of cardiac tissues except for the central zone of infarct. These results of phEPO gene therapy delivered by a bioreducible ABP polymer provide insight into the lack of phEPO gene therapy translation in the treatment of acute MI.

In an embodiment of the present invention, intramyocardial phEPO gene therapy delivered by the bioreducible ABP polymer demonstrates increased cardioprotective effects on post-infarct cardiac remodeling, compared with the treatment of rHuEPO protein and naked phEPO plasmid-alone. The prominent effects of phEPO/ABP gene therapy are accompanied by the preservation of cardiac geometry and function, reduction in the density of fibrotic tissue, protection against cardiomyocyte loss, decrease in apoptotic activity, stimulation of angiogenesis, inhibition of α-SMA+ myoFb differentiation, and suppression of the profibrotic Ang II and TGF-β expression across the LVfb and remote non-infarcted sites after MI.

In yet another embodiment, the favorable cardioprotective effects of phEPO/ABP polyplexes are not confined only to the LV infarct lesion, as with ACE-Is or ARBs. Rather, these effects can spread into non-infarcted sites including the IVS, RV, and atria. Functional, histopathologic, and molecular analysis between the well-known rHuEPO, phEPO-alone, and embodiments of the present phEPO/ABP delivery system has provided a deeper understanding of the subcellular remodeling process to help find an advanced therapeutic approach for MI. Unlike rHuEPO or phEPO treatment-alone, embodiments of the present phEPO gene therapy delivered by biodegradable ABP provides a surprisingly effective gene therapy to reverse post-infarct cardiac remodeling and eventually restore cardiac function. In some aspects, the amount of restoration achieved is to about the same level as a thoracotomy control with supporting mechanisms.

In one embodiment of the invention, administration of therapeutically effective amounts of a phEPO/ABP polyplex to a subject with a cardiac condition may provide a cardioprotective effect on cardiac remodeling that maintains cardiac function at nearly the same level as a level prior to the cardiac condition. In one embodiment, the level of cardiac function may be at least about 85% of the level of function prior to the cardiac condition. In another embodiment, the level of function may be at least about 95% of the level of function prior to the cardiac condition. In a further embodiment, the level of function may be at least about 98% of the level of function prior to the cardiac condition.

In one aspect of the invention, a cardioprotective polyplex is provided. Such a polyplex may include a pCMV-hEPO DNA (phEPO) complexed with an arginine-conjugated bioreducible poly(disulfide amine) polymer (ABP) having the structure:

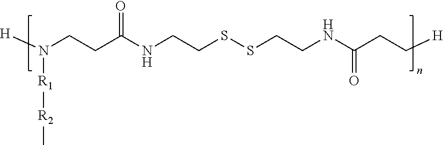

wherein n is about 1 to 1000 and wherein R1 is (CH2)mNH, wherein m is 1 to 18, and R2 is an arginine residue. In one aspect, m can be 6. In another aspect, n can be 4 to 8.

Physical and chemical properties of polyplexes encompassed by the present invention may vary. For example, in one aspect, the polyplex can have a particle size, or an average particle size when present as a plurality of particles, of from about 100 nm to about 500 nm. In another aspect, the size may be from about 150 nm to about 450 nm. In another aspect, the size may be from about 200 nm to about 300 nm. In yet another aspect, the size may be about 215 nm.

In one embodiment, a polyplex of the herein-recited type can have a zeta potential of from about 10 mV to about 40 mV. In another aspect, the zeta potential can be from about 20 mV to about 30 mV. In yet another aspect, the zeta potential may be about 28 mV.

Additionally, the components of the phEPO/ABP polyplex can be tuned or adjusted to arrive at a polyplex with a desired performance characteristic. In one aspect, the phEPO and ABP can be present in a weight ratio of from 1:1 to 1:40. In another aspect, the ratio can be from 1:1 to 1:20. In a further aspect, the ratio can be from 1:1 to 1:10. In yet an additional aspect, the ratio can be 1:5.

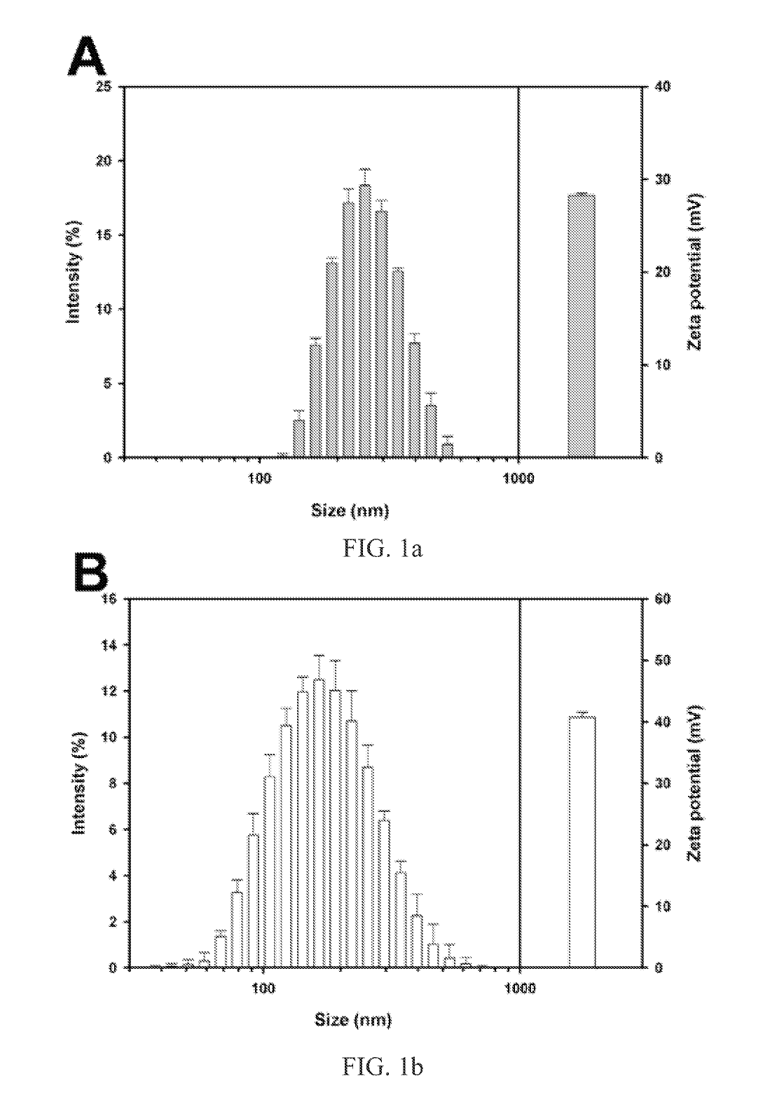

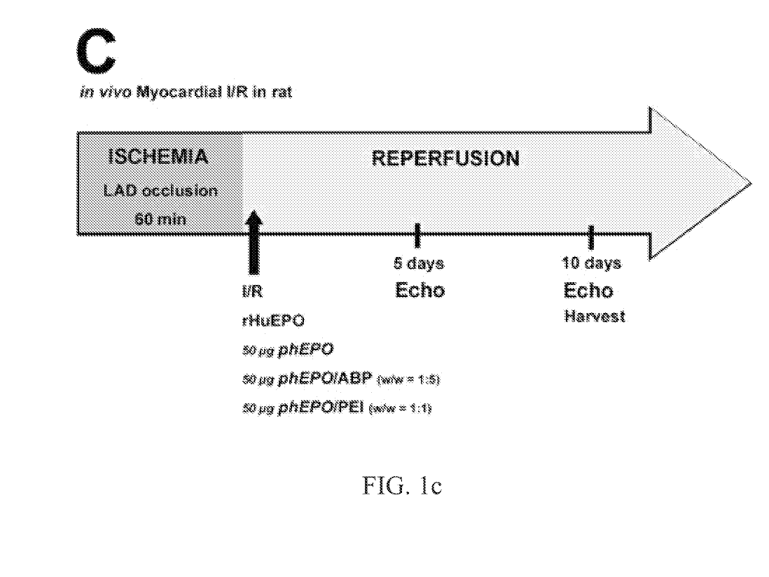

In one specific embodiment, a phEPO/ABP polyplex can have phEPO and ABP present in a weight ratio of 1:5 with an average particle size of about 214.6±3.7 nm and a zeta potential of about 28.3±0.2 mV. In another embodiment, the phEPO may have 4,578 bp. In a further aspect, the ABP polymer can have a molecular weight, or an average molecular weight when present as a group, of about 5K to about 50K or kDA. In another aspect, the weight may be about 25K. In a further aspect, the weight may be about 10K. In yet another aspect, the weight may be about 5K. In a further aspect, the phEPO/ABP polyplex can have a polydispersity index (PDI) of from about 0.08 to about 1.2. In yet another aspect, the PDI can be about 0.093. When present in a group, in one embodiment phEPO/ABP polyplexes encompassed by the present invention can have a size distribution pattern as shown in FIG. 1a, when prepared according to the methods recited herein.

In addition to the polyplexes disclosed and described herein, compositions, such as compositions for treatment of a cardiac condition, containing such polyplexes are encompassed by the present invention. In one embodiment, such a composition can include a polyplex as recited herein and a pharmaceutically acceptable carrier. In one embodiment, the carrier can be water. In another embodiment, the carrier can include a buffer. In a further embodiment the buffer can be glucose.

Such compositions may be selected to achieve specific dosage forms and/or accommodate specific routes of administration. In one embodiment, the composition is suitable for parenteral administration to a subject (i.e. parenteral dosage form). In another aspect, the dosage form may be suitable for systemic administration. In an additional aspect, the dosage form may be suitable for direct administration to cardiac tissue, for example, by intramyocardial injection. In further embodiments, the composition may be prepared so as to provide sustained a sustained or extended erythropoietic effect as compared to administration, including similar administration of equivalent amounts of naked (i.e. non-complexed) phEPO or rHuEPO.

In addition to the polyplexes and compositions described herein, the present invention encompasses methods for using such polyplexes and compositions. In one embodiment, a method for transfecting a cell, including a cardiac, kidney, or other cell capable of erythropoietin production, with phEPO can be performed. Such a method may include providing a phEPO/ABP polyplex, as recited herein, and contacting the cardiac cell with the polyplex. In some aspects, the transfection or contact may occur in-vitro and in some aspects, it may occur in-vivo. In one aspect, the cell can be a cardiomyocyte. In another aspect, the cell can be a kidney cell, including a renal peritubular cell. In yet another aspect, the cell can be a liver cell.

In another embodiment, a method for treating a condition in a subject which is responsive to erythropoietin therapy may be performed. In one embodiment, treatment may include providing a phEPO/ABP polyplex, or a composition containing such, and/or administering a therapeutically effective amount of the phEPO/ABP polyplex or composition containing such to the subject. Examples of conditions can include any disease, state, condition, or ailment which benefits, improves, or is ameliorated by an increase in erythropoietin presence, levels, or concentration. In one example, the condition may be a cardiac condition. In another example, the condition may be an inflammatory bowel disease, such as Crohn's disease and ulcer colitis. In yet a further example, the condition may be anemia or an underlying cause of anemia. In another example, the condition may be kidney disease, including chronic renal failure. In a further example, the condition may be a physiologic condition which negatively impact or reduces kidney performance, such as diabetes. In an additional example, the condition may be any disease, state, condition, or ailment, which causes or contributes to a reduction in volume, output, lifespan, or performance of red blood cells, including anemia associated cancers, or treatments for diseases, such as chemotherapy or radiation therapy. In yet a further example, the condition may be a hypoxia condition regardless of and including any underlying cause.

In one embodiment, a method for treating a cardiac condition in a subject can be performed. Exemplary conditions are myocardial infarction and cardiac remodeling, particularly deleterious cardiac remodeling, for example, that can occur following a myocardial infarction event. In one aspect, such a method may include administering a therapeutically effective amount of a polyplex as recited herein to the subject. Such administration can in some embodiments occur via presentation of a composition as recited herein to the subject. In some aspects, such administration can be parenteral and utilize a parenteral composition or dosage form. In another aspect, the administration can be systemic. In a further aspect, the administration can be localize do cardiac tissue, for example, by intramyocardial injection.

Administration of a phEPO/ABP polyplex to a subject using the polyplexes and compositions recited herein can provide an erythropoietic effect of an extended duration. In one aspect such a duration may be longer than a duration provided by an equivalent amount of naked (i.e. unbound) phEPO or rHuEPO with a same administration mechanism. In one aspect, the duration can be from about 10 minutes to about 60 days following administration. In another aspect, the duration can be for at least 6 hours following administration. In yet a further aspect, the duration can be for at least 4 hours following administration.

By administration of the phEPO/ABP polyplexes and compositions discussed and described herein, it has been discovered that cardiac function in a subject that has experienced myocardial infarction can be preserved. In one aspect, a method of preserving cardiac function in such a subject can include administering to the subject a therapeutically effective amount of a phEPO/ABP polyplex, or a composition containing such, to the subject. Timing of administration can be important. In one aspect, administration may occur within 24 hours of myocardial infarction. In another aspect, administration may occur within 8 hours of myocardial infarction. In yet another aspect, administration may occur within 1 hour of myocardial infarction.

Additionally, the present invention encompasses methods for controlling or directing cardiac remodeling in a subject suffering from a cardiac condition. Generally, such a method can include administering a therapeutically effective amount of a phEPO/ABP polyplex or a composition containing such to the subject.

In yet another embodiment, a method of suppressing AngII and TGF-β activity in cardiac tissue that has experienced a cardiac condition is provided. One example of such a method includes administering a therapeutically effective amount of a phEPO/ABP polyplex or a composition containing such to the cardiac tissue.

Likewise, a method of suppressing expansion of an infarct zone in acute myocardial infarction is provided and may include administering a therapeutically effective amount of a phEPO/ABP polyplex as recited herein to the infarct zone. Such a method can also apply to any type or form of cardiac condition or threat to cardiac tissue. In one aspect, administration to the infarct (or other threat) zone can occur within 4 hours of the commencement of infarct and/or threat. In another aspect, administration can occur within 1 hour of commencement of the infarct/threat. In yet a further aspect, administration to the infarct/threat zone can provide a cardioprotective effect on non-infarcted tissue remote from the infarct/threat zone. One example of such tissue is tissue adjacent to or surrounding the infarct/threat zone.

In addition to the foregoing, the present invention provides for use of a phEPO/ABP polyplex as recited herein in the preparation of a medicament for treatment of a cardiac condition. As previously noted, exemplary conditions to be treated include myocardial infarction and cardiac remodeling.

### EXAMPLES

The following examples are provided to promote a more clear understanding of certain embodiments of the present invention, and are in no way meant as a limitation thereon.

### Materials and Methods

Preparation of phEPO/Polymer Polyplexes

pCMV-hEPO DNA (phEPO) (4,578 bp) was constructed and purified as follows. The human erythropoietin (hEPO) cDNA was amplified by polymerase chain reaction using pDrive-hEPO (Open Biosystems, Huntsville, Ala.) as a template. The PCR primer sequences were as follows:

forward primer,
5′-CCGGAATTCATGGGGGTGCACGAATGTC-3′;
reverse primer,
5′-GCTCTAGATCATCTGTCCCCTGTCCTGCAG-3′.

The EcoRI and XbaI sites were introduced to the PCR primers for cloning. The amplified hEPO cDNA was purified by agarose gel electrophoresis and elution. The hEPO cDNA was inserted into pCI (Promega) at the EcoRI and XbaI sites, resulting in construction of pCMV-hEPO (phEPO). The proper construction of the pCMV-hEPO was confirmed by direct sequencing. The constructed phEPO was amplified in E. coli DH5α. phEPO and GFP pDNA (gWiz-GFP, Aldevron) were purified with an endotoxin-free plasmid DNA purification NucleoBond® Xtra Maxi plus EF kit (Macherey-Nagel Inc.). Purity and concentration of the purified plasmid dissolved in TE buffer were measured using a Nanodrop 1000 spectrophotometer, and the purities at A260/A280 were 1.8-1.9. Branched poly(ethylenimine) (bPEI, 25 kDa, Sigma-Aldrich) and rHuEPO protein (Aropotin®) were used as controls.

The arginine-modified bioreducible polymer, ABP, was synthesized by arginine modification into the primary amines of poly(CBA-DAH). The backbone poly (CBADAH) polymer was synthesized by Michael reaction of equivalent moles of and N,N′-cystaminebisacrylamide (CBA) and tert-Butyl-N-(6-aminohexyl)carbamate (N-Boc-1,6-diaminohexane, N-Boc-DAH) in methanol/water solution (9:1, v/v), and the polymerization reaction was maintained under a dark nitrogen atmosphere at 60° C. for 4 days. Then, 0.1 equivalent of N-Boc-DAH was added to terminate the polymerization by masking unreacted acrylamide groups and the reaction mixture was further stirred for 1 day at the same temperature. After the resulting product was precipitated with cold diethyl ether, Boc protecting groups of the product were removed by trifluoroacetic acid (TFA) solution (TFA:triisobutylsilane:water=95:2.5:2.5, v/v) for 30 min. After de-protection, the reaction mixture was precipitated with diethyl ether, dialyzed using a dialysis membrane (MWCO=1000, Spectrum Laboratories, Inc., Rancho Dominguez, Calif.) and then lyophilized. The synthesis of poly(CBA-DAH) was confirmed by 1H NMR spectra. Arginine coupling to the poly(CBA-DAH) was performed in dimethylformamide (DMF) for 2 days at room temperature with 4 equivalents of 2-(1H-benzotriazole-1-yl)-1, 1, 3, 3-tetramethyluronium hexafluorophosphate (HBTU), Fmoc-L-Arg(pbf)-OH and 8 equivalents of N,N-diisopropylethylamine (DIPEA), respectively. The reaction was monitored by ninhydrin test. After the completion of the arginine modification, the crude mixture was precipitated to remove the unreacted and excess reagents with cold ethyl ether. The reactant was deprotected with 30% piperidine solution (DMF, VAT) for Fmoc and 95% TFA solution for pbf groups. After precipitation with cold ethyl ether, the crude product was dialyzed against water with the dialysis membrane (MWCO=1000) followed by freeze drying. Arginine modification was confirmed with 1H NMR, and an average molecular weight was determined by size exclusion chromatography (SEC). The average molecular weight was found to be approximately ˜5 K.

The 100 μL of phEPO/ABP polyplex solutions (50 μg of phEPO) were prepared at the weight ratios (pDNA/polymer) of 1 to 5 in a 20 mM HEPES/5% glucose buffer. As control, 50 μg phEPO/PEI polyplexes solutions at the weight ratio of 1 to 1 in a 20 mM HEPES/5% glucose buffer were prepared. After 30 min incubation, polyplex solutions were diluted in double filtered water to a final volume of 600 μL before measurement. The average particle size and Zeta-potential values of the polyplexes were measured using a Nano ZS (ZEN3600, Malvern Instruments) with a He—Ne ion laser (633 nm). Graphical representations of the phEPO/ABP polyplex and phEPO/PEI polyplex are shown in FIGS. 1a and 1b respectively.

### Experimental Rats

Male Sprague-Dawley rats (from Charles River Laboratories) at 6-7 weeks of age were purchased. All rats were housed in accordance with Association for Assessment and Accreditation of Laboratory Animal Care International (AAALAC) guidelines. All experiments followed the guidelines provided by the National Institutes of Health in Guide for the Care and Use of Laboratory Animals and conformed to the American Heart Association guidelines for the use of animals in research. All rats had access to food and water ad libitum and were housed in plastic cages on standard 12/12 h light/dark cycles. The rats were randomly assigned to the one of eight groups: 1) sham thoracotomy, 2) I/R only, 3) rHuEPO protein injection, 4) human EPO plasmid DNA (phEPO) injection, 5) phEPO/ABP polyplex injection, 6) phEPO/PEI polyplex injection, and 7) GFP pDNA/ABP polyplex injection.

### Myocardial Infarction

As generally shown in FIG. 1c, myocardial infarction was induced in male Sprague-Dawley (SD) rats (7-8 weeks old with a body weight of 220-250 g) by surgical occlusion of left anterior descending (LAD) coronary artery. Briefly, the SD rats were anesthetized under 4% isoflurane for induction (VIP3000™ veterinary vaporizer, Midmark), intubated, and mechanical ventilation was maintained with a small-animal respirator (Harvard Apparatus) (tidal volume=12 ml/kg, respiratory rate=60 cycles/min) under 2% isoflurane for maintenance of anesthesia at 38° C. on the heating pad (T/Pump TP650, Gaymar industries Inc.). The left chest was shaved and a thoracotomy was performed in the 4th or 5th intercostal space, exposing the heart. The LAD coronary artery was ligated 2-3 mm from its origin with a single stitch of 6-0 prolene suture (Ethicon) under the 2.5 magnification (HiRes®, Surgical Acuity). The ligature ends were passed through a small length of plastic tube (PESO polyethylene tubing, Becton Dickinson) to form a snare. For coronary artery occlusion, the snare was pressed onto the surface of the heart directly above the coronary artery and a hemostat was applied to the snare. Successful ischemia was verified by the blanching of the myocardium and dyskinesia of the ischemic zone, indicating interruption in coronary flow. After 60 min of occlusion, the hemostat was removed, and the snare was released for reperfusion. Restoration of normal rubor indicated successful reperfusion of myocardium. Following successful ischemia-reperfusion (I/R), the animals were assigned to one of seven groups as previously mentioned, namely: 1) sham thoracotomy, 2) I/R only, 3) injection of rHuEPO, 4) injection of phEPO alone, 5) injection of phEPO/ABP polyplex, 6) injection of phEPO/PEI polyplex, and 7) injection of GFP plasmid/ABP polyplex. Right after reperfusion, the 3)-7) rats received total injection volume of 100 μl delivered to four separate intramyocardial sites (each 25 μl) with three injections to the ischemic border zone of the infarct and one injection to the central zone. After the injection, wounds were sutured in layers and the thorax was closed under negative pressure, chest tube thoracostomy. Additionally, some animals received a full thoracotomy with exposure of the heart, but no ligation of the LAD to act as sham operation controls (n=5). Animals were gradually weaned from the ventilator. Animals received analgesia (0.05 mg/kg buprenorphine 1M per 12 hrs) for 2 days and antibiotic prophylaxis (0.05 g cefazolin IP) for 5 days.

### Transthoracic Echocardiography

To evaluate the left ventricular geometry and function, two-dimensional guided M-mode images of trans-thoracic echocardiography were performed in short and long axis projections using a 13 MHz linear probe (GE Vivid 7 pro, GE Medical Systems) after 5 days and 10 days of intramyocardial injection as previously described. After myocardial I/R surgery (5 d, 10 d), rats were lightly anesthetized with isoflurane 1-2 L/min and spontaneous respiration, imaged in the right lateral decubitus position, and temperature was maintained at 37° C. on a heating pad (T/Pump TP650, Gaymar industries, Inc.). Next, the chest hair was removed with shaving and a topical depilatory agent. Left ventricular dimensions and wall thickness were measured in at least three beats from each projection and averaged. Fractional shortening [%] was calculated as [(LVDd−LVDs)/LVDd]*100 and ejection fraction [%] was calculated as [(LVDd3−LVDs3)/LVDd3]*100; where LVDd=left ventricular diastolic dimension and LVDs=left ventricular systolic dimension. In an apical long-axis view, pulsed wave doppler recordings were made with the sample volume placed in the left ventricular outflow tract (LVOT). Stroke volume [μl] was calculated as π*(LVOT diameter/2)2*LVOT VTI; where VTI=the velocity time integral [cm]. Cardiac output (C.O.) was calculated as SV*HR; where SV=stroke volume [μl] and HR=heart rate [beats/min].

### Organ Harvest and Pathological Analysis

On the same day of transthoracic echocardiography examination at postoperative 10 days, rats were sacrificed by overdose of isoflurane gas inhalation, and the hearts were excised. The heart serially flushed with phosphate buffered saline (PBS), heparin (5 unit/ml)-PBS to flush any remaining blood, 2.56M KCl solution to arrest it in diastole, and fixed in 10% formalin. The heart was sliced into 2 mm-thick transverse sections using a rodent heart slicer matrix (Zivic Instruments), dehydrated through an ascending ethanol series, and embedded in paraffin.

First, serial sections of 4 μm were cut and stained with H-E stain. Second, collagen in the heart sections was stained using Masson's trichrome. The infarct size of myocardium was calculated by the total infarction area divided by the total LV area using ImageScope (Aperio technologies Inc. Vista, Calif.). Collagen content is expressed as percentage collagen containing pixels per tissue section area. Third, immunohistochemical (IHC) staining was performed on the 4 μm thick sections of formalin-fixed, paraffin-embedded tissue. Sections were air-dried at room temperature and then placed in a 60° C. oven for 30 min to melt the paraffin. All of the staining steps were performed at 37° C. using an automated immunostainer (BenchMark® XT, Ventana Medical Systems). To evaluate the arteriolar density and the loss of cardiomyocytes after the myocardial infarction, heart sections were immunohistochemically stained using α-smooth muscle actin (α-SMA #M0851 monoclonal antibody, Dako) and cardiomyocyte specific troponin T (cTnT #T6277 monoclonal antibody, Sigma). The sections were detected using the IView DAB detection kit-research (Ventana Medical Systems), which is an open secondary, Streptavidin-HRP system, utilizing DAB (3-3′ diaminobenzidine) as the chromogen. The sections were counterstained with hematoxylin (Ventana Medical Systems) for 8 min.

Arterioles positive for α-SMA over the infarcted zone were counted in five random highpower fields (×10 magnification) using ImageScope (Aperio technologies Inc. Vista, Calif.) per whole heart pecimen. Arterioles were defined as vessels with an internal diameter of 10-50 μm. Counts from 30 microscopic fields were averaged and expressed as the number of capillaries and arterioles per hpf. The loss or recovery of cardiomyocytes by cTnT staining was determined throughout the transverse sections of heart specimen. The determination of apoptosis was performed using a commercially available kit (ApopTag Apoptosis Detection Kit, Intergen). Slides were mounted and observed with a confocal microscope with a ×20 objective. Apoptosis in the infarcted regions was expressed as the number of terminal transferase-dUTP-Nick End Labeled (TUNEL) positive nuclei per unit area and examined in five random high power fields (hpfs) per section. For all the histology every hpf was randomly chosen within the infarct border zone by an investigator blinded to the treatment groups. Analysis of all images was carried out with NIH Image software (NIH, Bethesda, Md.) and Aperio ImageScope (Vista, Calif.).

### Western-Blot Analysis of Ang II and TGF-β

On the post-infarct 10 days, rats were anesthetized with isoflurane, and hearts were harvested, separated, and weighed into the infarct area and border zone of LV wall, interventricular septum, RV, and atrium. Samples were immediately snap-frozen in liquid nitrogen and stored at 80° C. until analysis. About 50 μg of heart tissue was homogenized in 200 μl lysis buffer (50 mmol/L Hepes, 150 mmol/L NaCl, 10% Glycerol, 1% Triton X-100, 1.5 mmol/L MgCl2, 1 mmol/L EGTA, 10 mmol/L Sodium Pyrophosphate, 100 mmol/L Sodium Fluoride and 100 μmol/L Sodium Vanadate, 1 mmol/L PMSF, 10 μg/ml Aprotinin, and 10 μg/ml Leupeptin) using a Mini-Beadbeater™ (Biospec products Inc.) and centrifuged for 20 min at 13,500 rpm at 4° C. After normalizing for the protein concentration of the lysate supernatants by bicinchoninic acid (BCA) assay (Pierce® BCA protein assay kit, Pierce biotechnology), 20 μg of protein was separated using 4-20% SDS-polyacrylamide gel electrophoresis (PAGE) (Mini-Protean® TGX™ Precast gel, Bio-rad) and transferred onto immun-Blot® poly-vinylidene difluoride membrane (PVDF, Bio-rad) and blocked for 1 h at room temperature in blocking solution (filtered 5% BSA (Cohn fraction V) in TBST). The immunoblots were incubated with agitation at 4° C. overnight in the presence of specific antibodies directed against Ang II (1:500) (#251229 rabbit polyclonal, Abbiotec) and TGF-β (1:1000) (#3711 rabbit polyclonal, Cell Signaling Technology) in filtered 1% BSA in TBST. After washing in TBST solution, the blots were further incubated for 1 h at room temperature with horseradish peroxidaseconjugated secondary antibody (1:2000) (#7074 anti-rabbit, Cell Signaling Technology) and streptacin-HRP conjugate (Precision Protein™ StrepTactin-HRP conjugate, Bio-rad) in filtered 1% BSA in TBST. The blots were then washed three times in TBST, and antibodybound protein was visualized with the Enhanced Chemiluminescence (ECL) kit (Immun-Star™ WesternC™ chemiluminescent kit, Bio-rad). The α-Actin (1:1000) (#A2172 mouse monoclonal antibody, Sigma-Aldrich) was used as a housekeeping protein for the purposes of normalization, followed with secondary antibody (#7076 anti-mouse, Cell Signaling Technology). Signals were quantified by molecular imager ChemiDoc XRS (Bio-rad) and densitometric analysis was normalized to anti-Actin (α-sarcomeric).

### Statistical Analysis

Data for the results below is expressed as the mean±SD or mean±SEM where indicated. Comparisons between multiple groups were performed by analysis of variance (ANOVA) followed by Tukey post hoc testing. Groups with P values less than 0.05 were considered statistically significant.

### Results

Referring to FIGS. 1a and 1b, it can be seen, the 50 μg phEPO/ABP polyplexes at a weight ratio of 1:5 showed an average particle size of 214.6±3.7 nm and a zeta potential of 28.3±0.2 mV. The polydispersity index (PDI) and size distribution pattern of phEPO/ABP polyplexes are homogeneously condensed (PDI=0.093) as compared to 50 μg phEPO/PEI polyplex (w/w=1:1) (PDI=0.162).

Beyond the conventional erythropoietic activity, EPO is also thought to be a pleiotropic organ-protective cytokine. Because of the resolution of myocardial stunning and reperfusion injury, the LV ejection fraction (LVEF), a powerful prognostic parameter improves steeply during the first week after MI. The effect of intramyocardial phEPO/ABP polyplex injections on the time-dependent LV remodeling of cardiac geometry and function using echocardiography examination, compared with other treatment groups in post-infarcted hearts was evaluated. The general protocol is shown in FIG. 1c.

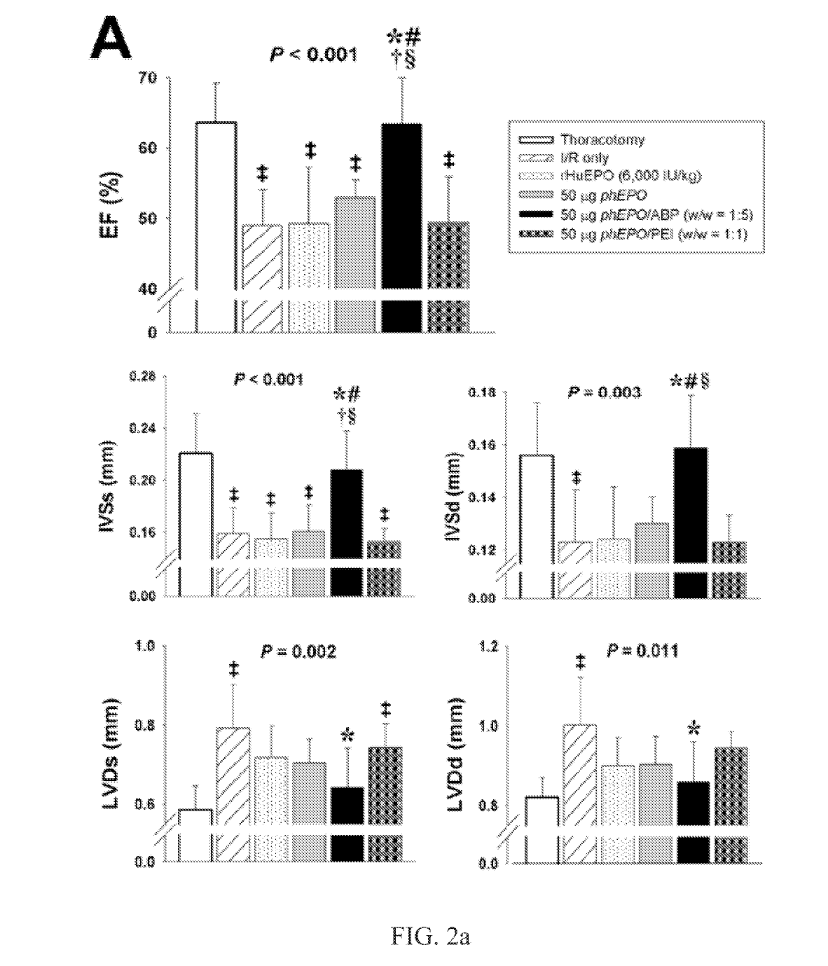

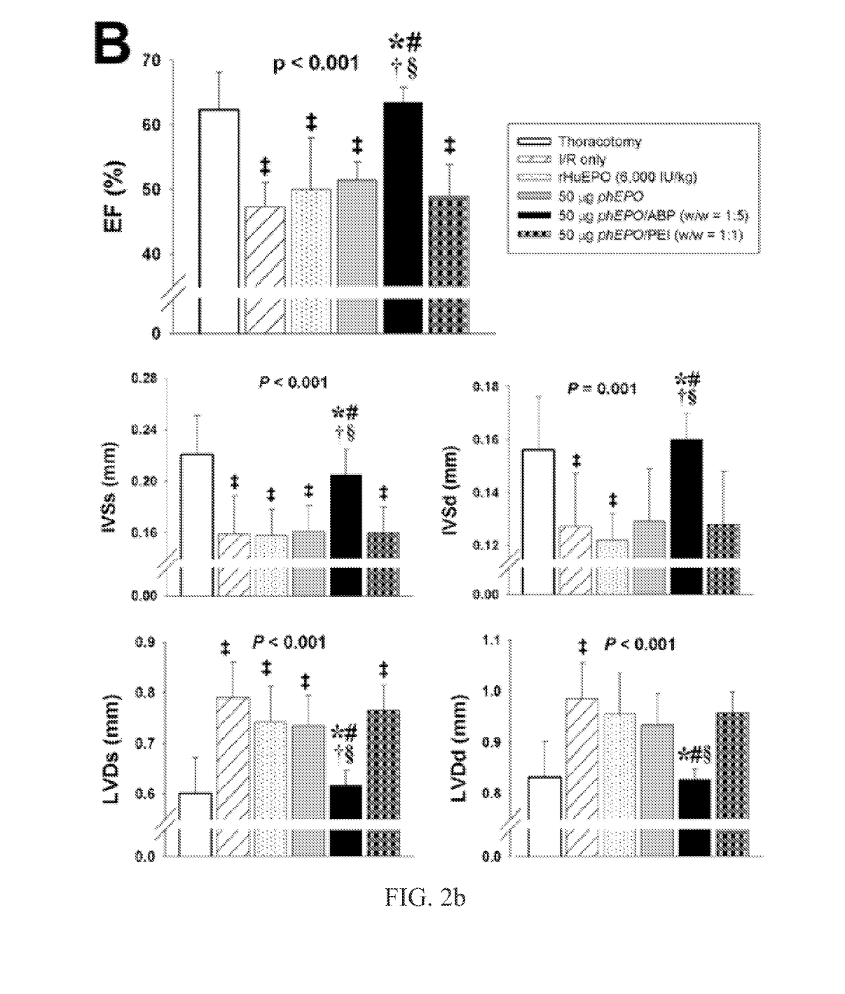

On both post-infarct days 5 and 10, the administration of phEPO/ABP polyplexes showed an improved LVEF comparable up to the level of sham thoracotomy group and a significantly preserved LVEF when compared with other treatment groups as shown in FIGS. 2a and 2b. An LVEF increase of >3% with the administration of β-blockers and ACE-inhibitors has shown reduced morbidity and mortality in patients with acute MI. The observed ˜15% improvement of the ejection fraction in the phEPO/ABP group, reaching the reference level of the thoracotomy group is interesting.

As a result, the present inventors conclude that phEPO/ABP conserves cardiac geometry and function during post-infarct cardiac remodeling. Referring again to FIGS. 2a and 2b, it can be seen that the thickness of interventricular septum during systole (IVSs), thickness of interventricular septum during diastole (IVSd), left ventricular diameter during systole (LVDs), left ventricular diameter during diastole (LVDd) is very favorable when the phEPO/ABP polyplexes of the present invention are administered. Specifically, FIGS. 2a and 2b show that the hpEPO/ABP polyplexes achieved the following: □P<0.05 vs. thoracotomy, *P<0.05 vs. I/R group, #P<0.05 vs. rHuEPO, †P<0.05 vs. phEPO-alone group, §P<0.05 vs. phEPO/PEI group.

Referring again to FIGS. 2a and 2b, is can be seen that administration of phEPO/ABP attained a conserved IVS thickness during the systolic (IVSs) and diastolic phase (IVSd), nearly up to the level of the thoracotomy group on both post-infarct days 5 and 10 than other treatment groups. In addition, the LV diameters during the systolic (LVDs) and diastolic phase (LVDd) for the phEPO/ABP injection group were remarkably reserved to the level of the thoracotomy group on post-infarct day 10. The post wall thickness of the LV during the systolic and diastolic phase did not reveal any differences between the groups. All of the echocardiographic parameters of the GFP DNA/ABP polyplex injection group were comparable to the I/R-only group, excluding the impact of the ABP polymer itself. Collectively, these results show that phEPO gene therapy delivered by the ABP polymer improves the cardiac geometry and LV systolic function during post-infarct cardiac remodeling, especially acting on the IVS and preventing LV dilation. In the phEPO/ABP group, the conserved hemodynamic alterations and LV dimension may result in a more favorable prognosis after infarct, preventing post-infarct HF.

In addition to the foregoing, administration of phEPO/ABP ameliorates cardiac fibrosis with a reduced infarct size. In the heart, myocardial fibrosis following the loss of myocardial muscle mass is a common pathological end point including additional MI. Initially, fibrosis through increased interstitial collagens is beneficial to the heart by preventing ventricular dilation. However, the cumulative deposition of collagen results in reduced cardiac function with increased stiffness, and post-infarct morbidity, such as HF. The present evaluation assessed whether the administration of phEPO/ABP polyplexes during I/R injury had an effect in the suppression of cardiac fibrosis on post-infarct cardiac remodeling by the decrease in collagen contents.

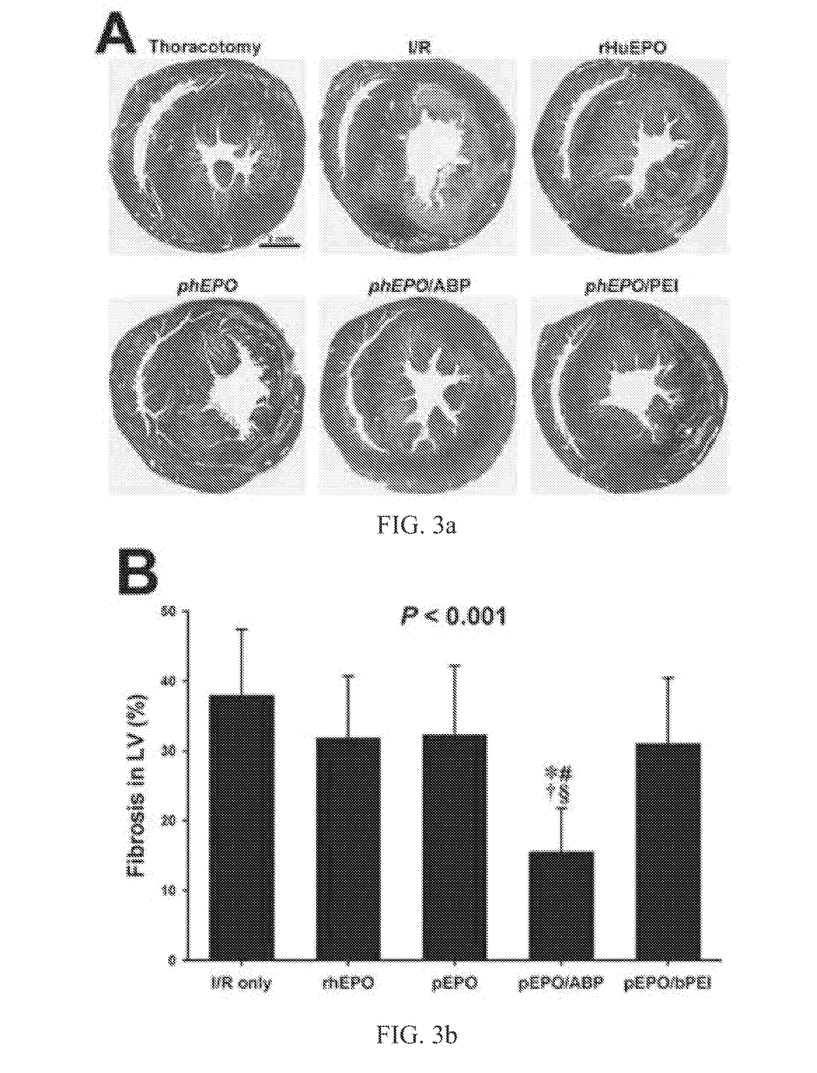

Referring now to FIG. 3a, it can be seen that upon Masson's trichrome staining, the post-infarct fibrotic scar areas with bluish-stained high-collagen contents in the LV were decreased in the phEPO/ABP polyplex injection group as compared to the I/R group (15.6±6.2% vs 38.0±9.4%; P<0.01. As shown in FIG. 3b, quantitative analysis revealed that % fibrosis of phEPO/ABP group is significantly decreased compared with other groups. This decreased fibrosis may account for at least a portion of the preserved functional effects of the phEPO/ABP polyplex injection in the post-infarct heart compared with other treatment groups. A lowering of myocardial fibrosis of up to 60% by the phEPO/ABP polyplexes in the infarcted LV eventually suggests alleviate chamber stiffness, halting adverse cardiac remodeling. Specifically, FIGS. 3a and 3b show that the hpEPO/ABP polyplexes achieved the following: (mean±SD; n=4-6 per group). *P<0.01 vs. I/R group, # P<0.01 vs. rHuEPO, †P<0.01 vs. phEPO-alone group, §P<0.01 vs. phEPO/PEI group.

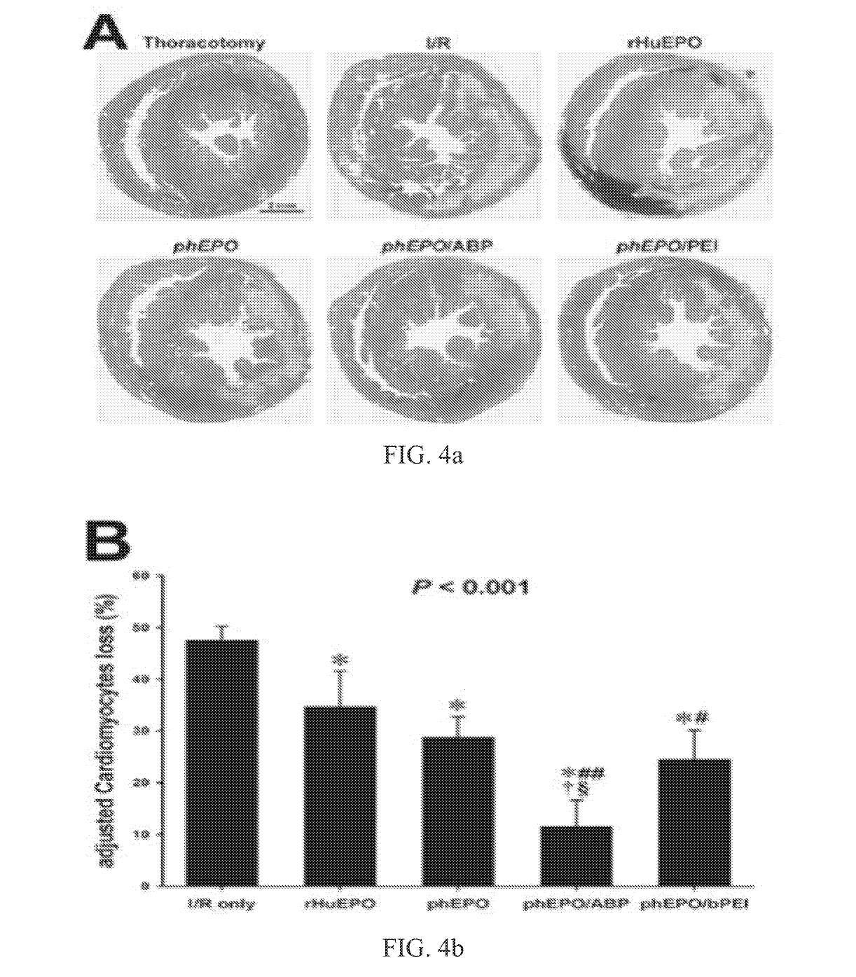

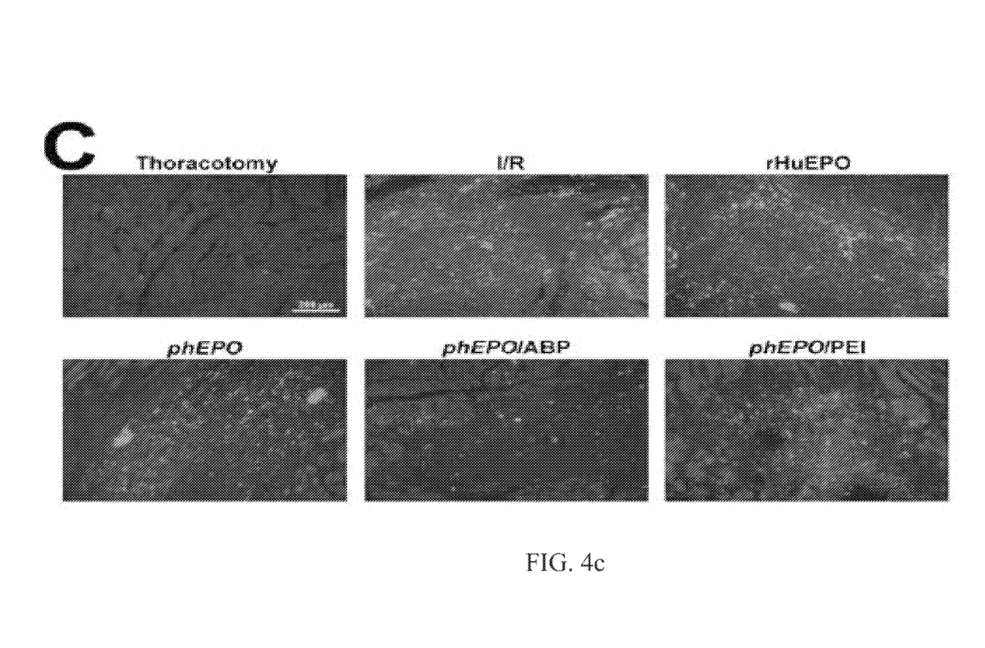

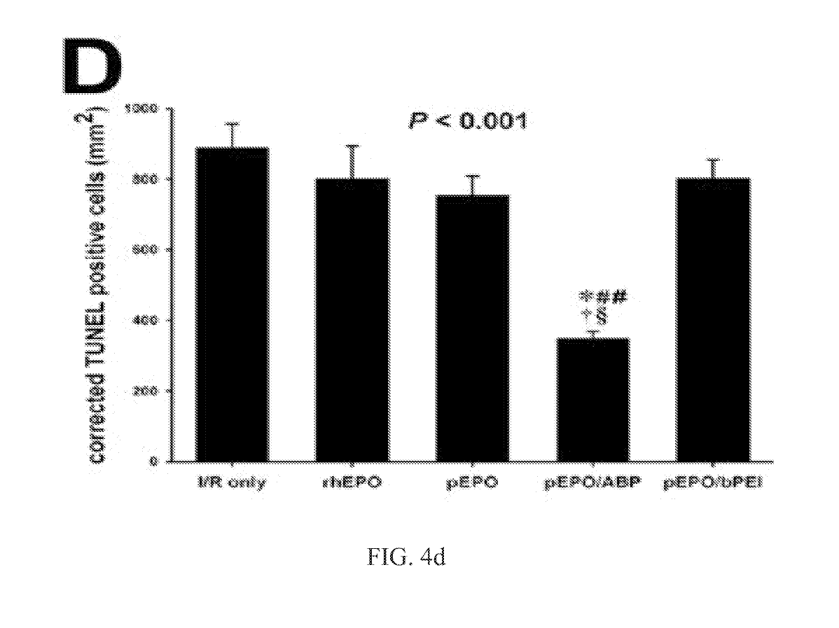

Referring now to FIGS. 4a-4d it can be seen that administration of phEPO/ABP preserves cardiomyocyte loss and provides lower apoptotic activity. The ongoing cardiomyocytes loss from necrosis or apoptosis is one of the early pathological characteristics in MI. As such, the efficacies of the different treatments with regard to the loss of cardiomyocytes 10 days after MI were evaluated as shown in FIGS. 4a and 4b. In the results of cTnT immunohistochemical staining, the adjusted percent of cardiomyocytes lost was significantly elevated in all of the treatment groups—rHuEPO, phEPO, phEPO/ABP, and phEPO/PEI—as with the I/R-only group (P<0.01; FIG. 4B). Compared to the rHuEPO group, the phEPO/ABP (P<0.01) and phEPO/PEI polyplex groups (P<0.05) showed significantly decreased cardiomyocytes loss. Taken together, only the phEPO/ABP group showed significantly preserved cardiomyocyte numbers compared with other treatment groups (P<0.001). During post-infarct cardiac remodeling, reperfusion injury results in the paradoxical acceleration of apoptosis in the reperfused myocardium.

The degree to which the administration of phEPO/ABP inhibits the apoptotic activity in the LVfb was also evaluated by comparison to other groups. The apoptotic activity measured by TUNEL staining revealed lower apoptosis in the phEPO/ABP polyplex injection group (348.4±145.3/mm2) than that of other groups. Consistent with previous results, stronger inhibition of apoptosis in the phEPO/ABP treatment group diminished infarct size, favoring improvement in cardiac function after MI. Referring again to FIGS. 4a-4d, it is shown that administration of phEPO/ABP minimizes cardiomyocytes loss and apoptotic activity 10 days after MI by at least the following: 4a—representative IHC staining images for cTnT in the mid-ventricle of hearts from each group (n=4-6). Bar=2 mm; 4b—quantification of percent cardiomyocytes loss in LV adjusted by the level of thoracotomy group (mean±SD; n=4-6 per group); 4c—representative TUNEL staining images in the LVfb from each group. Bar=200 μm; and 4d—quantification of corrected TUNEL positive cells (mm2) corrected by the level of thoracotomy group (mean±SEM; n=4-6 per group). *P<0.01 vs I/R group, #P<0.05 vs rHuEPO, ##P<0.01 vs rHuEPO, †P<0.01 vs phEPO-alone group, §P<0.01 vs phEPO/PEI group.

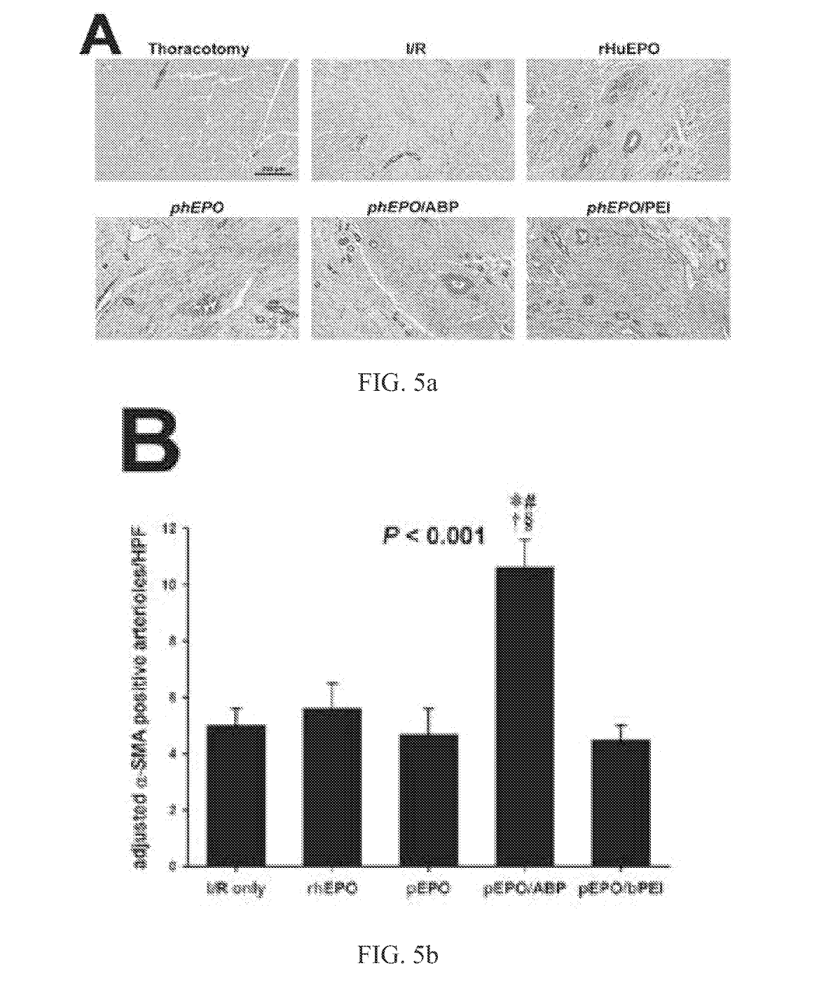

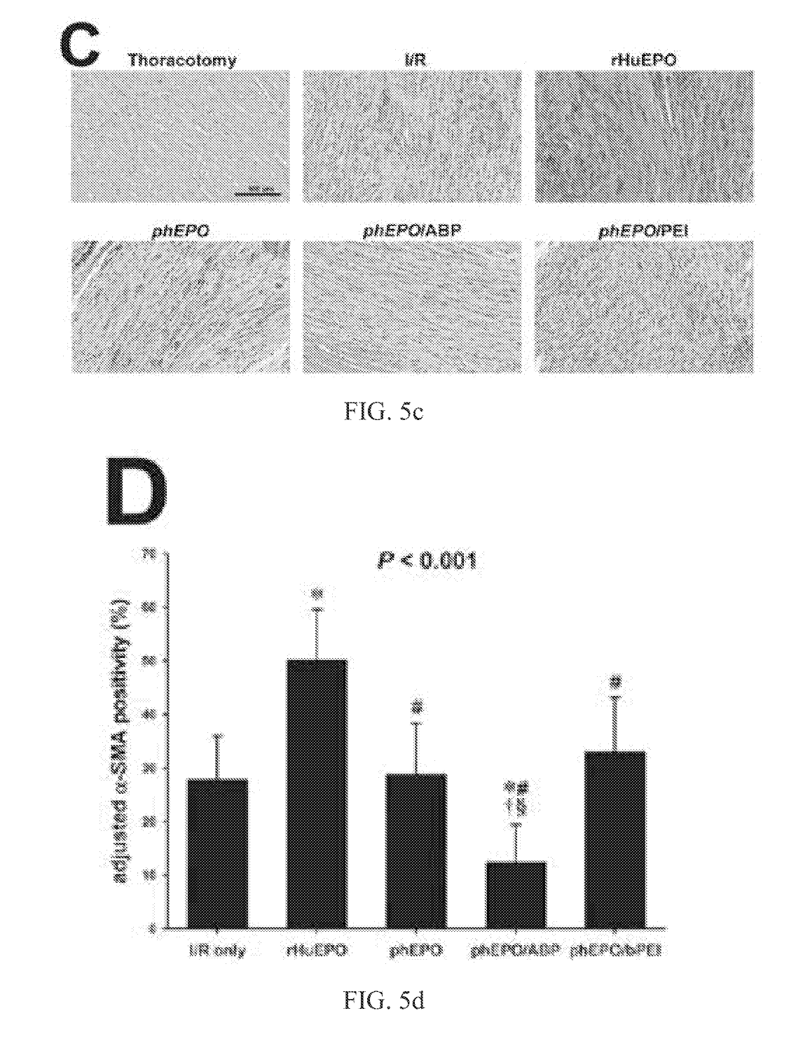

Additionally, phEPO/ABP administration can enhance angiogenesis and modulate the activation of myoFbs. During the healing phase of post-infarct cardiac remodeling, the blood supply to the infarcted myocardium is restored by angiogenesis and by remodeling of the vascular tree to conserve cardiac function. Referring now to FIGS. 5a-d, it can be seen that IHC staining for α-SMA showed more abundant arterioles in the phEPO/ABP polyplex injection group than in other treatment groups. The mean number of α-SMA-positive arterioles per hpf increased from 5.0±0.6 in the I/R only group to 10.6±1.0 in the phEPO/ABP polyplex injection group (P<0.01; FIGS. 5A and 5B). The administration of the phEPO/ABP polyplexes revealed a higher upregulation of angiogenic activity in the LVfb than other treated groups, which could increase capillary-to-myocyte ratio, decrease the oxygen diffusion distance, and consequently improve oxygen supply to the infarcted myocardium.

During cardiac remodeling, the activated myofibroblasts (myoFb, collagen-secreting novo α-SMA+-expressing fibroblasts) replace the lost cardiomyocytes and form nonregenerative scar tissue by depositing profibrotic molecules such as collagen and fibronectin in the extracellular matrix. MyoFb is the predominant source of collagen mRNA in healing MI, which has the characteristics of fibroblasts and smooth muscle cells, has at least a twofold stronger contractile activity compared with α-SMA-negative fibroblasts, and eventually determines the infarct size and quality of the scar. MyoFb's are present 4-6 days after an infarction and peak with maximum proliferation within the first two weeks after acute MI in humans. Referring to FIGS. 5c and 5d, is shown testing results regarding the potent cardioprotective mechanism of phEPO/ABP as a function of the distribution and density of myoFbs, as shown by α-SMA expression in post-infarct cardiac remodeling between the different groups. As can be seen, there was comparable in α-SMA positivity for the phEPO-alone group and phEPO/PEI group compared with the I/R group.

The analysis of the adjusted α-SMA expression in the LVfb highlights the distinct differences between the treatment groups. In particular, the rHuEPO group and the phEPO/ABP group represent two extremes of α-SMA activity in the LVfb. The phEPO/ABP group had up to a 75% decrease in α-SMA expression compared to that measured in the rHuEPO group and a 55% decrease compared to the I/R group (P<0.01; FIG. 5D). The exaggerated activation of myoFbs after post-infarct cardiac remodeling is significantly modulated in the phEPO/ABP group compared with the other treatment groups. In the rHuEPO group, increased myoFbs may form fibrotic scars, preventing infarct expansion, ventricular dilation, and cardiac rupture. In addition, through their contractile activity, increased myoFbs generate tensile strength, helping the function of the infarcted heart. Collectively, the enhanced myoFb density in the extracellular matrix of the rHuEPO group contributes to the salutary effects of rHuEPO administration to compensate for postinfarct cardiac remodeling. However, the persistent and excessive activation of myoFbs in the rHuEPO group with the consequent collagen production causes deleterious cardiac remodeling and unfavorable outcomes, such as fibrosis, contracture, and heart failure. On the contrary, the phEPO/ABP group modulated the spread and abundance of myoFbs by controlling α-SMA-expressing myoFb differentiation, accompanied by the conservation of cardiomyocyte loss. This entirely different characteristic of the phEPO/ABP group may induce favorable anti-remodeling effects in the infarcted heart. From this viewpoint, we could weigh in on the analysis of myoFb infiltrations between the treatment groups.

Referring again to FIGS. 5a-5d, it is shown that administration of phEPO/ABP increases angiogenesis (A, B) and modulation of fibroblast differentiation (C, D) in the LVfb according to different treatments 10 days after MI. FIG. 5a shows representative IHC staining images. Bar=200 μm. FIG. 5b shows quantification of pro-angiogenic activity by the α-SMA-positive arterioles adjusted by the level of thoracotomy group. FIG. 5c shows representative interstitial IHC staining images. Bars=100 μm. FIG. 5d shows quantification of α-SMA-positive myoFb differentiation adjusted by the level of thoracotomy group (mean±SD; n=4-6 per group). *P<0.01 vs. I/R, #P<0.01 vs. rHuEPO, †P<0.01 vs. phEPO-alone group, §P<0.01 vs. phEPO/PEI group.

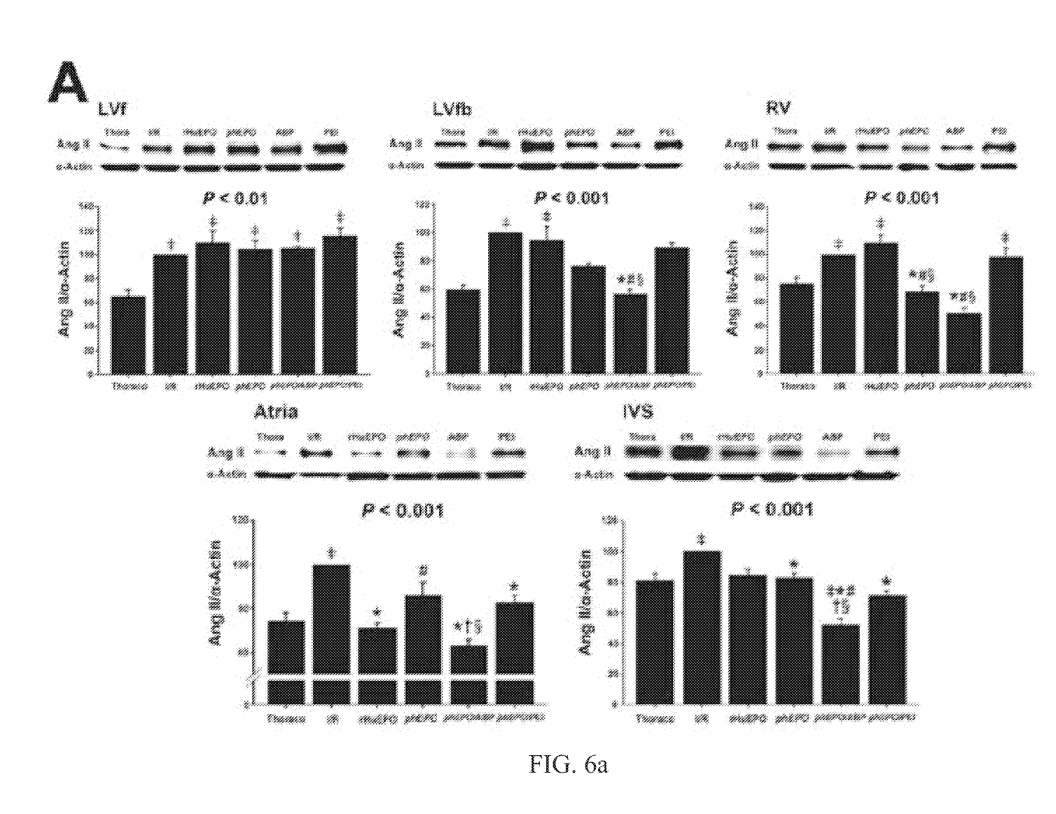

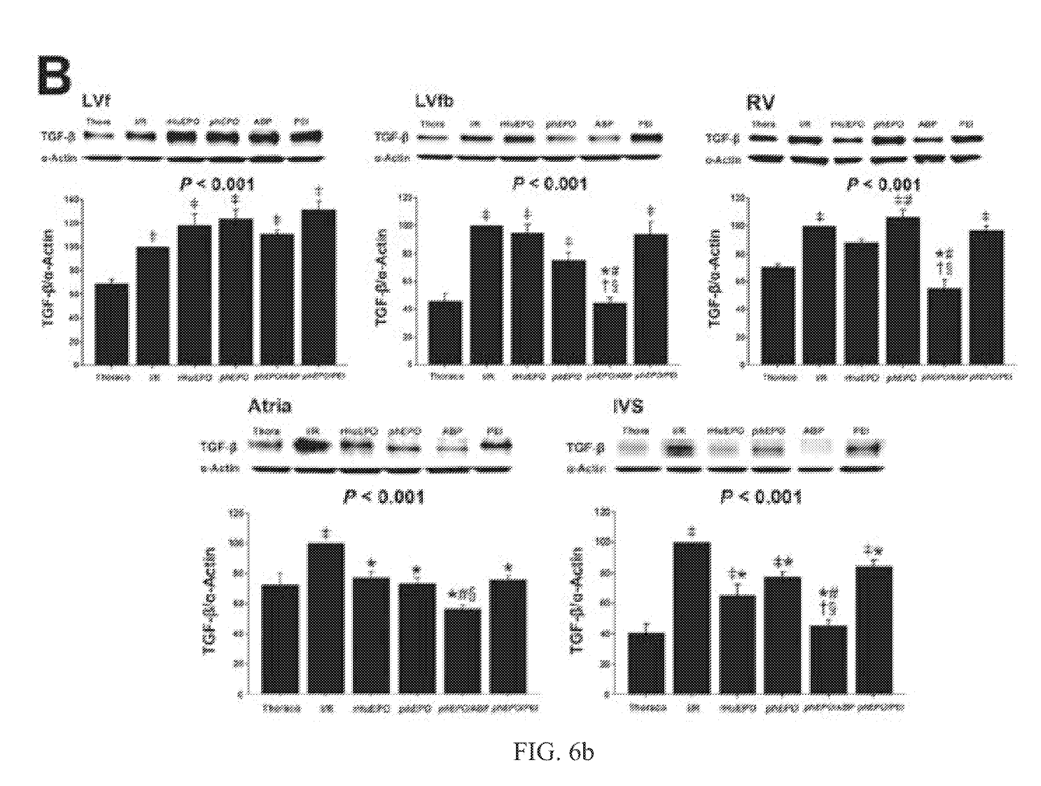

As shown in FIGS. 6a and 6b, administration of phEPO/ABP in accordance with the present invention suppresses pro-fibrotic Ang II effects on the heart. Neurohormones, such as Ang II and other inflammatory cytokines have functionally significant cross-talk, converging on common signal transduction pathways in cardiac remodeling after MI. Especially, the beneficial actions of the renin-angiotensin system (RAS) blockers making an impact upon patient survival are better correlated with the inhibition of tissue RAS levels rather than plasma levels. Activation of the local cardiac tissue RAS, with its regulation independent of the systemic RAS, has important physiological and pathological roles, including post-infarct cardiac remodeling. Beyond its regulation of blood pressure and fluid homeostasis, Ang II—the final physiologically active effector of RAS—has multiple effects on the heart, inducing myocyte apoptosis/necrosis and inflammation, driving perivascular fibrosis and scarring, stimulating fibroblast proliferation and collagen deposition, and inducing differentiation of cardiac fibroblasts into myoFbs. Blockers of RAS are clinically well-proven treatments in patients with MI, preventing LV remodeling and eventually improving survival. Independent of their blood pressure-lowering effect, widely prescribed ACE-inhibitors and ARBs are able to reverse the extent of myocardial fibrosis, reduce the LV chamber stiffness, and improve the LV function by pleiotropic and additional off-target effects on cardiac fibroblasts of the remodeling heart.

A number of underlying molecular mechanisms may explain the potential effects phEPO/ABP gene delivery, compared with other treatment groups. After I/R, Ang II expression increased in a whole subdivision of cardiac tissues (P<0.05; FIG. 6A). The suppression of Ang II expression in the phEPO/ABP group reached comparable levels to that of the thoracotomy group in the LVfb, RV, and atria, and it was at an even lower level than the thoracotomy group in the IVS (FIG. 6A). Compared with the I/R group, phEPO/ABP gene delivery showed remarkable decreases in Ang II in all of the cardiac tissues excluding the LVf (P<0.05; FIG. 6A). The phEPO/ABP group had significantly lower Ang II expression than that of the rHuEPO group in the LVfb, RV, and IVS (P<0.05; FIG. 6A); than that of the phEPO group both in the atria and IVS. Collectively, compared with the rHuEPO and phEPO-alone group, the phEPO gene therapy delivered by the ABP polymer demonstrated a significant suppression of pro-inflammatory and pro-fibrotic Ang II expression in the periinfarct as well as at non-infarcted remote sites (IVS, RV, and atria), implying stronger and more far-reaching effects on post-infarct cardiac remodeling. However, in the LVf, all treatment groups failed to suppress Ang II expression.

In addition to the foregoing, phEPO/ABP reduces fibrogenic TGF-β activity on the heart. TGF-β is a major cytokine that both initiates and terminates tissue repair, and its sustained production underlies cardiac hypertrophy by interstitial fibrosis and phenotypic differentiation of cardiac fibroblasts to α-SMA+ myoFbs, causing the transition from an inflammatory to a proliferative phase during infarct healing. TGF-β1 expression is upregulated in infarcted regions and in patients with fibrotic disorders. Ang II directly stimulates TGF-β1 production, thus initiating cardiac fibrosis during the transition from stable hypertrophy to heart failure with the upregulation of fibronectin and collagen genes, and blockade of the TGF-β signaling pathway results in significant amelioration of deleterious post-MI cardiac remodeling with down-regulation of the RAS. Expression levels of TGF-β were analyzed according to the anatomical division between the groups. TGF-β expressions were increased in all subdivisions of cardiac tissues after myocardial I/R (P<0.05; FIG. 6B). Particularly in the IVS, the entire treatment group demonstrated a significant suppression of TGF-β expression compared with the I/R group (P<0.05; FIG. 6B). The suppression of TGF-β expression in the phEPO/ABP group reached levels comparable to that of the thoracotomy group in the LVfb, RV, atria, and IVS, except for in the LVf (FIG. 6B). This decreased expression of TGF-β in the phEPO/ABP group within the peri-infarct as well as the remote zones explains the complementary functional and histologic favorable anti-remodeling effects. These combined findings illustrate that the phEPO/ABP group mitigates post-infarct cardiac fibrosis by preventing collagen-secreting α-SMA+ myoFb differentiation through the inhibition of Ang II and TGF-β.

Together with Ang II expression, measurement of TGF-β levels in cardiac anatomical subdivisions elucidated that EPO itself was insufficient to reverse the fibrosis-dominated disease process, like the LVf during cardiac remodeling. In addition, the relatively increased activity of Ang II and TGF-β in the rHuEPO and phEPO injection-alone groups accounts for a portion of the increased metabolic activity of the enhanced myoFbs. Therefore, the sustained release or expression of EPO modified by the delivery system—and not the short acting administration of rHuEPO or phEPO—is able to protect against the cardiac ischemic cascade at histological and molecular levels.

Accordingly, as shown in FIGS. 6a and 6b, administration of phEPO/ABP more effectively modulates Ang II and TGF-β expression in cardiac tissues as compared to other treatment groups. This activity is shown by the Western blot analysis according to the subdivision of cardic tissues—LVf, LVfb, RV, atria, and IVS. Representative image of Western blots and quantitation of Ang II (A) and TGF-β (B) expression in myocardial tissue (mean±SEM; n=4-7 per group). ABP, 50 μg phEPO/ABP (w/w=1:5); PEI, 50 μg phEPO/PEI (w/w=1:1); □P<0.05 vs. thoracotomy, *P<0.05 vs. I/R group, #P<0.05 vs. rHuEPO, †P<0.05 vs. phEPO-alone group, §P<0.05 vs. phEPO/PEI group.

In view of the foregoing results, without wishing to be bound by theory, the present inventors note a number of underlying mechanisms that could contribute to the amount and degree of cardioprotection from phEPO/ABP delivery after MI. First, we it has been observed that phEPO/ABP polyplexes protected pDNA from degradation in vitro for over 6 hours in the presence of serum, which allows for an increased circulation time in vivo. The characteristics of the bioreducible ABP carrier may contribute to prolonged release and circulation times of the loaded phEPO gene. By contrast, it is well known that the naked pDNA is not stable in blood and is degraded within minutes after intravenous injection and therefore the instability of phEPO in the blood likely reduces its effect on the cardiac remodeling process. Second, under clinical and anatomical backgrounds, the compact extracellular matrix of the myocardium filled with negatively charge molecules such as glycosaminoglycan and proteoglycan may be a major drawback for cardiac gene delivery, especially for positively charged particles, compared to neutralized and negatively charged naked plasmid DNA and siRNA-—alone. This effect was apparent in the absence of efficacy displayed by the highly cationic PEI polyplex control group. Third, direct injection of pDNA itself into the cardiac muscle results in 10-100 times more efficiency of gene expression than injection of the same amount of pDNA into skeletal muscle. The treatment route of intramyocardial local injection may amplify the cardioprotective effect of the phEPO/ABP gene therapy. Fourth, inflammation is one of the main pathophysiologic mechanisms in postinfarct cardiac remodeling. The in vivo innate immune response measured by the plasma IL-6 levels was comparable between the phEPO-alone and phEPO/ABP polyplex groups, even with a higher amount of phEPO and ratio of phEPO/ABP. Fifth, the favorable pathologic findings of lessened fibrosis, and reduced necrosis in the phEPO/ABP polyplex group possibly allow the delivered phEPO gene to remain in the intact extracellular matrix of the post-infarcted heart to transfect viable cells. Sixth, the average size and distribution of the particles are important factors to determine the pharmacokinetics and pharmacodynamics of the delivered drug and gene. The phEPO/ABP polyplexes had a more condensed homogenous distribution than the phEPO/PEI polyplexes. Seventh, the superiority of the ABP polyplexes in cardiac remodeling may be explained by the well-known toxicity limitation of the PEI polymer, offsetting its positive biological effects.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

What is claimed is:

1. A cardioprotective polyplex comprising:
a pCMV-hEPO DNA (phEPO) complexed with an arginine-conjugated bioreducible poly(disulfide amine) polymer (ABP) having the structure: 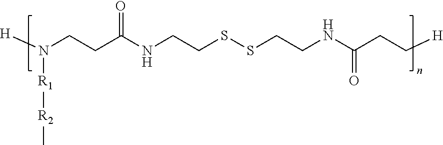
wherein n is about 1 to 1000 and wherein R1 is (CH2)mNH, wherein m is 1 to 18, and R2 is an arginine residue.

2. The polyplex of claim 1, wherein the polyplex has a particle size of from about 100 nm to about 500 nm.

3. The polyplex of claim 1, wherein the polyplex has a zeta potential of about 28 mV.

4. The polyplex of claim 1, wherein the phEPO and ABP are present in a weight ratio of from 1:1 to 1:40.

5. The polyplex of claim 1, wherein the phEPO and ABP are present in a weight ratio of 1:5 with an average particle size of about 214.6±3.7 nm and a zeta potential of about 28.3±0.2 mV.

6. The polyplex of claim 1, wherein the phEPO has 4,578 bp.

7. The polyplex of claim 1, wherein m is 6.

8. The polyplex of claim 1, wherein n is 4 to 8.

9. The polyplex of claim 1, wherein the ABP polymer has an average molecular weight of about 5K.

10. The polyplex of claim 1, wherein the polyplex has a polydispersity index (PDI) of about 0.093.

11. The polyplex of claim 1, wherein the polyplex has a size distribution pattern as shown in FIG. 1a.

12. A composition for treatment of a cardiac condition in a subject comprising:
a therapeutically effective amount of a polyplex as recited in any of claims 1-11; and
a pharmaceutically acceptable carrier.

13. The composition of claim 12, wherein the carrier is water.

14. The composition of claim 12, further comprising a buffer.

15. The composition of claim 14, wherein the buffer is glucose.

16. The composition of claim 12, wherein the composition is suitable for parenteral administration to the subject.

17. The composition of claim 16, wherein the parenteral administration is systemic.

18. The composition of claim 16, wherein the parenteral administration is intramyocardial.

19. The composition of claim 16, wherein the polyplex provides sustained erythropoietic effect as compared to administration of equivalent amounts of naked phEPO or rHuEPO.

20. A method for transfecting a cardiac cell with phEPO, comprising:
providing a polyplex as set forth in any of claim 1-11, and
contacting the cardiac cell with the polyplex.

21. The method of claim 20, wherein the contacting occurs in vitro.

22. The method of claim 20, wherein the contacting occurs in vivo.

23. The method of claim 20, wherein the cell is cardiomyocyte.

24. A method for treating a cardiac condition in a subject comprising:
administering a therapeutically effective amount of a polyplex as recited in any of claims 1-11 to the subject.

25. The method of claim 24, wherein administration is parenteral.

26. The method of claim 25, wherein the parenteral administration is systemic.

27. The method of claim 25, wherein the parenteral administration is localized to cardiac tissue.

28. The method of claim 27, wherein the administration is intramyocardial.

29. The method of claim 25, wherein the administration provides an erythropoietic effect for a duration that is longer than a duration provided by an equivalent amount of naked phEPO or rHuEPO with a same administration mechanism.

30. The method of claim 25, wherein the duration is from about 10 minutes to about 60 days following administration.

31. The method of claim 30, wherein the duration is for at least 6 hours following administration.

32. The method of claim 30, wherein the duration is for at least 4 hours following administration.

33. The method of claim 25, wherein the cardiac condition is myocardial infarction.

34. The method of claim 25, wherein the cardiac condition is cardiac remodeling.

35. A method of preserving cardiac function in a subject that has experienced myocardial infarction, comprising:
administering to the subject a therapeutically effective amount of a phEPO/ABP polyplex as recited in any of claims 1-11.

36. The method of claim 35, wherein the administration occurs within 24 hours of myocardial infarction.

37. The method of claim 36, wherein the administration occurs within 8 hours of myocardial infarction.

38. The method of claim 37, wherein the administration occurs within 1 hour of myocardial infarction.

39. A method of controlling cardiac remodeling in a subject suffering from a cardiac condition comprising
administering a therapeutically effective amount of a phEPO/ABP polyplex as recited in any of claims 1-11 to the subject.

40. A method of suppressing Ang II and TGF-β activity in cardiac tissue that has experienced a cardiac condition comprising:
administering a therapeutically effective amount of a phEPO/ABP polyplex as recited in any of claims 1-11 to the cardiac tissue.

41. A method of suppressing expansion of an infarct zone in acute myocardial infarction comprising;
administering a therapeutically effective amount of a polyplex as recited in any of claims 1-11 to the infarct zone.

42. The method of claim 41, wherein administration to the infarct zone occurs within 4 hours of the commencement of infarct.

43. The method of claim 42, wherein administration to the infarct zone occurs within 1 hour of commencement of infarct.

44. The method of claim 41, wherein administration to the infarct zone provides a cardioprotective effect on non-infarcted tissue remote from the infarct zone.

45. The method of claim 44, wherein the non-infarcted tissue is adjacent to the infarct zone.

46. Use of a phEPO/ABP polyplex as recited in any of claims 1-11 in the preparation of a medicament for treatment of a cardiac condition.

47. The use of claim 46, wherein the cardiac condition is myocardial infarction.

48. The use of claim 46, wherein the cardiac condition is cardiac remodeling.
